(19) United States
(12) Patent Application Publication
(10) Pub. No.: US 2017/0157163 A1
(43) Pub. Date: Jun. 8, 2017

(54) METHOD FOR TREATING, STABILIZING OR SLOWING DOWN BRAIN GLUCOSE METABOLISM DEFICIT

(71) Applicant: N.V. Nutricia, Zoetermeer (NL)

(72) Inventors: Maria Ladislaus BROERSEN, Utrecht (NL); Robert Johan Joseph HAGEMAN, Utrecht (NL); Marieke LANSBERGEN, Utrecht (NL); Martine GROENENDIJK, Utrecht (NL); Mattheus Cornelis DE WILDE, Utrecht (NL)

(73) Assignee: N.V. Nutricia, Zoetermeer (NL)

(21) Appl. No.: 15/115,477

(22) PCT Filed: Feb. 2, 2015
(86) PCT No.: PCT/NL2015/050064

§ 371 Date: Jul. 29, 2016

(30) Foreign Application Priority Data
Jan. 31, 2014 (NL) PCT/NL2014/050059

(51) Int. Cl. A61K 31/7068; A61K 31/14; A61K 31/4415; A61K 31/375; A61K 31/714; A61K 33/04; A61K 31/519; A61K 51/04; A61K 31/683; A61K 31/7072; A61K 31/202; A61K 31/355

(52) U.S. Cl. CPC A61K 31/7068; A61K 31/202; A61K 31/14; A61K 31/4415; A61K 31/375; A61K 31/355; A61K 33/04; A61K 31/519; A61K 51/0491; A61K 31/683; A61K 31/7072; A61K 31/714

(57) ABSTRACT

The invention pertains to the use of (i) one or more of uridine and cytidine, or salts, phosphates, acyl derivatives or esters thereof, and (ii) a lipid fraction comprising at least one of docosahexaenoic acid (22:6; DHA), eicosapentaenoic acid (20:5; EPA) and docosapentaenoic acid (22:5; DPA), or esters thereof in the manufacture of a composition for use in a method for treating, stabilizing or slowing down brain glucose metabolism deficit in a subject in need thereof.

The invention is in the field of medical nutrition and more particularly relates to a composition for treating, stabilizing or slowing down compromised brain glucose metabolism in subjects in need thereof.

### BACKGROUND OF THE INVENTION

The brain is one of the most metabolically active organs in the body and it uses glucose as a primary fuel for energy generation. This dependence on glucose puts the brain at risk if the supply of glucose is interrupted, or if cerebral glucose metabolism becomes defective. This is for instance the case for diminished cerebral glucose metabolism (DCGM, also known as glucose hypometabolism).

In the art there is thus the need to identify, prevent and treat impaired or reduced cerebral glucose metabolism.

### SUMMARY OF THE INVENTION

To that end, the inventors believe that brain function deterioration could be delayed or minimized by treating, stabilizing or slowing down brain glucose metabolism deficits in subjects in need thereof, by administering subjects in need thereof a product comprising (i) one or more of uridine and cytidine, or salts, phosphates, acyl derivatives or esters thereof, and (ii) a lipid fraction comprising at least one of docosahexaenoic acid (22:6; DHA), eicosapentaenoic acid (20:5; EPA) and docosapentaenoic acid (22:5; DPA), or esters thereof.

The brain glucose metabolism in the subject is readily monitored using Positron Emission Tomography with 18F-fluorodeoxyglucose (18F-FDG-PET).

It is preferred that the product further comprises at least one of (iii) choline, or salts or esters thereof; or (iv) at least one vitamin B selected from the group of vitamin B6, vitamin B12 and vitamin B9, or equivalents thereof, preferably comprising vitamin B6, B9 and B12. The product preferably comprises at least one of (iii) choline, or salts or esters thereof; and (iv) at least one vitamin B selected from the group of vitamin B6, vitamin B12 and vitamin B9, or equivalents thereof, preferably comprising vitamin B6, B9 and B12.

### LIST OF PREFERRED EMBODIMENTS

In one aspect, the invention pertains to the use of (i) one or more of uridine and cytidine, or salts, phosphates, acyl derivatives or esters thereof, and (ii) a lipid fraction comprising at least one of docosahexaenoic acid (22:6; DHA), eicosapentaenoic acid (20:5; EPA) and docosapentaenoic acid (22:5; DPA), or esters thereof, in the manufacture of a composition for use in a method for treating, stabilizing or slowing down brain glucose metabolism deficits in a subject in need thereof. Alternatively, the invention pertains to a method for treating, stabilizing or slowing down brain glucose metabolism deficits in a subject in need thereof, said method comprising administering to said subject a composition comprising (i) one or more of uridine and cytidine, or salts, phosphates, acyl derivatives or esters thereof, and (ii) a lipid fraction comprising at least one of docosahexaenoic acid (22:6; DHA), eicosapentaenoic acid (20:5; EPA) and docosapentaenoic acid (22:5; DPA), or esters thereof. Alternatively, the invention pertains to a composition for use in a method for treating, stabilizing or slowing down brain glucose metabolism deficits in a subject in need thereof, said composition comprising (i) one or more of uridine and cytidine, or salts, phosphates, acyl derivatives or esters thereof, and (ii) a lipid fraction comprising at least one of docosahexaenoic acid (22:6; DHA), eicosapentaenoic acid (20:5; EPA) and docosapentaenoic acid (22:5; DPA), or esters thereof.

In a preferred embodiment, said method involves identifying a subject in need thereof and/or monitoring brain glucose metabolism of said subject using Positron Emission Tomography with 18F-fluorodeoxyglucose (18F-FDG-PET).

In one embodiment, the subject is suffering from mild cognitive impairment (MCI) for instance assessed by having a mini-mental state examination (MMSE) of 20-26, preferably 21-26, more preferably 24-26, and/or a prodromal dementia or AD subject, exhibiting a level of more than 350 ng Total-tau per litre cerebrospinal fluid (CSF); and/or a weight ratio of abeta-42/Phospho-tau-181 of less than 6.5 in CSF.

In one embodiment, the subject identified as having an impaired cerebral glucose metabolism is not diagnosed with MCI or prodromal dementia or AD according to the above tools. The subject is preferably an elderly person. By identifying those subjects suffering from impaired or reduced cerebral glucose metabolism long before any diagnosis for Alzheimer's Disease [AD] or dementia is made, and providing such subjects with the intervention diet according to the invention, the onset of cognitive dysfunction and/or other dementia/AD-associated symptoms could be delayed.

In a preferred embodiment, the subject suffering from cerebral glucose metabolism deficit is a subject suffering from subjective memory concerns or subjective memory complaints (SMI) and/or subjects having a familial history of dementia or AD, said subject not suffering from MCI or prodromal dementia, prodromal AD. The subject is preferably an elderly person.

In the context of the present invention, a subject not suffering from a certain disorder may be interpreted as a subject not being diagnosed with said disorder.

In the above embodiments, the composition preferably further comprises (iii) choline, or salts or esters thereof; and/or (iv) at least one vitamin B selected from the group of vitamin B6, vitamin B12 and vitamin B9, or equivalents thereof. The composition preferably comprises vitamin B6, B9 and B12. Most preferably, the composition comprises at least uridine or UMP, and further DHA, choline, B6, B9 and B12.

### DETAILED DESCRIPTION OF THE INVENTION

With “treating, stabilizing or slowing down brain glucose metabolism deficit” it is understood that the (reduced) brain glucose metabolism is increased by diet intervention. The deficits may be assessed or monitored using Positron Emission Tomography with 18F-fluorodeoxyglucose (18F-FDG-PET). In one aspect, the brain glucose metabolism deficit is diminished cerebral glucose metabolism (DCGM) or commonly referred to as glucose hypometabolism.

Positron Emission Tomography with 18F-fluorodeoxyglucose (18F-FDG-PET) is a neuroimaging technique that provides a measure of cerebral glucose metabolism, which has been used in the field as an indicator of synapse function. Decreased 18F-FDG uptake, as assessed by 18F-FDG-PET, indicates decreased glucose metabolism in the brain and has been recognized as a stable and valid multicentre biomarker of synaptic dysfunction. Reduced glucose metabolism is first seen in the posterior cingulate and parieto-temporal cortices and involves frontal areas in later phases of AD.

The method may involve identifying or diagnosing brain glucose metabolism abnormalities using 18F-FDG-PET. In one embodiment, the method may involve monitoring brain glucose metabolism (impairments) using 18F-FDG-PET (discontinuously; at certain points in time) before and during intervention. These methods may involve the further step of comparing the brain glucose levels with those observed with the average reference situation (with no intervention). Progress could thus be monitored. The ‘identification’ may involve screening a subject using 18F-FDG-PET, determining brain glucose levels of said subject, comparing the brain glucose levels of said subject with an average reference situation, and assessing any differences. The assessment may lead to the diagnosis of impaired cerebral glucose metabolism, and consequently such diagnosis may lead to enrolling the subject in an intervention program wherein the product of the invention is administered to said subject. The product details and the preferred administration regime are described here below.

Throughout the application, in the context of the invention the wording “brain glucose metabolism” and “cerebral glucose metabolism” are used interchangeably. With ‘brain glucose metabolism deficits’ it is understood ‘compromised brain glucose metabolism’ or ‘brain glucose metabolism abnormalities’, which are readily assessed by skilled artisan. Abnormalities in cerebral glucose metabolism can be assessed by either taking the average values for glucose metabolism determined correspondingly a healthy subject (of similar age) (so matched for e.g. age; not diagnosed with any neurological disorder) as a reference. By comparison of the subject's condition with the reference situation. In particular, in the context of the invention deficits or abnormalities in glucose metabolism imply a decrease in brain glucose metabolism of preferably at least 10%, more preferably at least 15%, most preferably at least 20%, compared to the value as determined correspondingly in a healthy individual (of similar age). The value of x for the determination of cerebral glucose metabolism is preferably reduced by at least 10%, more preferably 15%, more preferably at least 20% when determined under standardized conditions in terms of feeding and exercise. These abnormalities could be assessed using FDG-PET (a decreased FDG signal) as detailed before, which is particularly suited to target specific brain areas in both the subject and the reference.

In one embodiment, the subject in need of the composition according to the invention is suffering from Alzheimer's Disease [AD] or other dementias such as vascular dementia or frontotemporal dementia, Parkinson's Disease, particularly said subject suffers from AD.

In one embodiment of the invention, the subject targeted is a human subject that has not been diagnosed with dementia or dementia-like disorders, but is suffering from mild cognitive impairment (MCI) or is a prodromal dementia subject or prodromal AD subject. A “prodromal dementia subject” is a person who does not suffer from a senile dementia, but has an increased likelihood to develop senile dementia. Likewise, a “prodromal AD subject” is a person who does not suffer from AD, but has an increased likelihood to develop AD. Here below, ‘prodromal subject’ refers to a prodromal AD or dementia subject. By providing subjects which suffer from impaired brain glucose metabolism, i.e. subjects which, in the absence of effective treatment, have a high probability to develop into all kinds of brain function impairment and neurodegenerative disorders such as dementia, in particular Alzheimer's disease (AD) with the product of the invention, it is believed that such brain function impairment and the onset of cognitive deficit symptoms could be delayed significantly.

In one embodiment, the prodromal AD or dementia subject is suffering from cognitive impairment according to the criteria from the National Institute on Aging and the Alzheimer's Association (NIA-AA) (Albert et al., Alzheimer's & Dementia. 2011; 7:270-279) or diagnosed as having mild dementia due to AD according to the NIA-AA criteria (McKhann et al., Alzheimer's & Dementia. 2011; 7:263-269. In one embodiment, the prodromal subject exhibits a level of more than 350 ng Total-tau per litre cerebrospinal fluid (CSF); and/or a weight ratio of abeta-42/Phospho-tau-181 of less than 6.5 in CSF. The prodromal subject preferably fulfils both requirements (total tau and ratio abeta-42/P-tau-181). Further explanations of the significance of concentrations of T-tau, P-tau-181 and Abeta42 in CSF for future development of Alzheimer's disease can be found in: Hansson O, Zetterberg H, Buchhave P, Londos E, Blennow K, Minthon L (2006) Association between CSF biomarkers and incipient Alzheimer's disease in patients with mild cognitive impairment: a follow-up study. Lancet Neurol 5:228-234; and in Pratico D, Clark C M, Liun F, Lee V Y M, Trojanowski J Q (2002) Increase in brain oxidative stress in mild cognitive impairment: a possible predictor of Alzheimer disease. Arch Neurol 59:972-976. In a preferred embodiment, the identification of prodromal subjects according to the invention comprises at least the first two criteria (total tau and ratio abeta-42/P-tau-181). In one embodiment, the method may involve identifying a subject being a prodromal AD or dementia subject, for instance using the above CSF biomarkers and/or 18F-FDG-PET.

In one embodiment, the subject may suffer from mild cognitive impairment (MCI). Reduced glucose metabolism (decreased 18F-FDG uptake) has been observed in subjects with MCI, which was greater in subjects with MCI who converted to AD or dementia later in life. MCI is a very common syndrome in elderly people and has a multitude of causes. Even though around 40-60% of subjects with the syndrome develop Alzheimer's disease during the first 5 years, many have a stable form or memory impairment. The subject suffering from MCI may be assessed as having a mini-mental state examination (MMSE) of 20-26, preferably 21-26, more preferably 24-26. The Mini Mental State Examination (MMSE) is the most commonly used test for complaints of memory problems, and as such used by practitioners in the field the MMSE questionnaires help the skilled person to assess changes in a person who has been diagnosed with dementia, but it can also help to give an indication of how severe a person's symptoms are and how quickly their dementia is progressing. On average, people with Alzheimer's disease who are not receiving treatment lose two to four MMSE points each year. In one embodiment, the method may involve identifying a subject being an MCI subject (with increased likelihood of developing AD or dementia), for instance using the above MMSE screening test.

In one preferred embodiment, the subject suffering from impaired cerebral glucose metabolism is a subject suffering from subjective memory concerns, subjective memory complaints and/or subjects having a familial history of dementia or AD, but for which subject no mild cognitive impairment has been assessed (which could be a subject having a MMSE score of 26 or higher). The subject may or may not score positively in the present diagnostic tools, preferably using the above CSF biomarkers (total tau and ratio abeta-42/P-tau-181). Reduced glucose metabolism may occur in cognitively normal elderly at clinical or genetic risk for AD and in cognitively normal elderly who progressed to MCI and AD, many years preceding the onset of dementia, and the above groups are at increased risk of developing dementia or AD.

In a preferred embodiment, the subject suffering from cerebral glucose metabolism deficit is a subject suffering from subjective memory concerns or subjective memory complaints (SMI) and/or subjects having a familial history of dementia or AD, said subject not suffering from MCI or prodromal dementia, prodromal AD. The subject is preferably an elderly person, preferably a person of the age of 50 or more, in particular of the age of 55 or more, more in particular of the age of 60 or more, more in particular of the age of 65 or more.

Within the group of subjects, preferably elderly, not [yet] assessed with MCI or prodromal dementia or AD, it was found that subjects having a familial record of dementia or AD are at increased risk of developing dementia or AD, and the composition of the invention is particularly suited for treating, stabilizing or slowing down cerebral glucose metabolism abnormalities in these subjects, particularly at the above age ranges. Within this group, it is particularly preferred to treat subjects with brain glucose metabolism abnormalities who show a maternal history of dementia or AD, as this subgroup has an even higher likelihood of developing dementia or AD. These subjects are known to suffer from a significantly reduced FDG-PET signal.

Within the group of subjects, preferably elderly not [yet] assessed with MCI or prodromal dementia or AD, yet suffering from brain glucose metabolism abnormalities, it was found that subjects suffering from subjective memory concerns, subjective memory complaints, sometimes referred to as SMI subjects, are also at increased risk. These are groups of subjects with self-expressed concerns are identifiable and classified in the art, for instance with the use of questionnaires. In order to be assessed as a subject having subjective memory concerns (‘SMI’), the subject could be asked to answer the question “Do you feel like your memory is becoming worse?” in a memory clinic for workup of memory impairment [possible answers: “no”; “yes, but this does not worry me”; and “yes, this worries me”, with the answer: “yes, this worries me.” (see Jessen et al. “AD dementia risk in late MCI, in early MCI, and in subjective memory impairment” Alzheimer's & Dementia 10 (2014) 76-83, its contents herein incorporated by reference). SMI is typically assessed in a longitudinal comparison (involving repeated observations of the same variables over long periods of time in the individual), and is defined by subjective reports of memory worsening and cognitive performance in the normal adjusted range. Epidemiologic studies have shown that SMI is a risk factor for cognitive decline and dementia, preceding MCI and prodromal stages. See also Scheef et al. “Glucose metabolism, gray matter structure and memory decline in subjective memory impairment” Neurology 2012; 79(13): 1332-1339, its contents herein incorporated by reference.

The inventors believe that 18F-FDG-PET is a reliable predictor of rapid progression to dementia even at the early stages where MCI and an increased likelihood of developing dementia or AD have not been diagnosed. Advantageously, with such early identification and intervention the onset of MCI, prodromal dementia or AD and eventually dementia or AD could be delayed. In a preferred embodiment, the method according to the invention involves identifying the subject suffering from brain glucose metabolism impairment having an increased likelihood of developing AD or dementia later in life, said subject at identification not being diagnosed having MCI or prodromal dementia or AD (i.e. having a MMSE of 26 or higher). The identification is based on the observation that the FDG uptake is decreased in such subjects compared to the average reference situation.

Preferably, the subject is drug-naïve, i.e. not on medication dedicated to the treatment of cognitive dysfunction or symptoms thereof. Subjects diagnosed with a neurodegenerative disorder such as dementia or AD are thus excluded. These diseases are presently diagnosed by a clinician by careful interpretation of a range of symptoms, as defined in the Diagnostic and Statistical Manual of Mental Disorders (fourth Edition, 2000)—DSM-IV-TR) or in McKhann, et al., Report of the NINCDS-ARDA-workgroup, Neurology 1984, 34, 939-944. The effects on glucose metabolism are considered particularly pronounced during the early pre-clinical stages.

The product of the invention is an enteral composition, intended for oral administration. It is preferably administered in liquid form. In one embodiment, the product comprises a lipid fraction and at least one of carbohydrates and proteins, wherein the lipid composition provides between 20 and 50 energy % of the food product. In one embodiment, the food product is a liquid composition containing between 0.8 and 1.4 kcal per ml.

### UMP

The present composition comprises uridine, cytidine and/or an equivalent thereof, including salts, phosphates, acyl derivatives (e.g. C₁₋₆ acylated uridine) and/or esters (e.g. C₁₋₆ alkanoate ester). In terms of uridine, the composition preferably comprises at least one uridine or an equivalent thereof selected from the group consisting of uridine (i.e. ribosyl uracil), deoxyuridine (deoxyribosyl uracil), uridine phosphates (UMP, dUMP, UDP, UTP), nucleobase uracil and acylated uridine derivatives. In one embodiment, cytidine, CMP, citicoline (CDP-choline) may also be applied. Preferably, the present composition comprises an uridine phosphate selected from the group consisting of uridine monophosphate (UMP), uridine diphosphate (UDP) and uridine triphosphate (UTP); and/or a cytidine phosphate (CMP, CDP, CTP, preferably CMP). Most preferably the present composition comprises UMP, as UMP is most efficiently being taken up by the body. Preferably at least 50 weight % of the uridine in the present composition is provided by UMP, more preferably at least 75 weight %, most preferably at least 95 weight %. Doses that must be administered are given as UMP. The amount of uracil sources can be calculated taking the molar equivalent to the UMP amount.

The present method preferably comprises the administration of uridine (the cumulative amount of uridine, deoxyuridine, uridine phosphates, nucleobase uracil and acylated uridine derivatives) in an amount of (i) 0.1 to 6 g per day, preferably 0.2 to 3 g per day, more preferably 0.4 to 2 g per day, and/or (ii) 0.1 to 6 g per 100 ml (liquid) composition, preferably 0.2 to 3 g per 100 ml (liquid) composition, more preferably 0.4 to 2 g per 100 ml (liquid) composition. In one embodiment, the above amounts also account for any amounts of cytidine, cytidine phosphates and citicoline incorporated in the composition or method. Uridine and its equivalents are however preferred.

Preferably, the present composition comprises uridine phosphate, preferably uridine monophosphate (UMP). The UMP is very efficiently taken up by the body. Hence, inclusion of UMP in the present composition enables a high effectivity at the lowest dosage and/or the administration of a low volume to the subject.

### DHA/EPA

The composition comprises at least one ω-3 polyunsaturated fatty acid (LC PUFA; having a chain length of 18 and more carbon atoms) selected from the group consisting of docosahexaenoic acid (22:6; DHA), eicosapentaenoic acid (20:5; EPA) and docosapentaenoic acid (22:5 ω-3; DPA), preferably at least one of DHA and EPA. Preferably the present composition contains at least DHA, more preferably DHA and EPA. EPA is converted to DPA (ω-3), increasing subsequent conversion of DPA to DHA in the brain. Hence, the present composition preferably contains a significant amount of EPA, so to further stimulate in vivo DHA formation.

The DHA, EPA and/or DPA are preferably provided as triglycerides, diglycerides, monoglycerides, free fatty acids or their salts or esters (e.g. C₁₋₆ alkyl ester), phospholipids, lysophospholipids, glycerol ethers, lipoproteins, ceramides, glycolipids or combinations thereof. Preferably, the present composition comprises at least DHA in triglyceride form.

In terms of daily dosage, the present method preferably comprises the administration of 500 to 5000 mg DHA+EPA+DPA (preferably DHA+EPA) per day, more preferably 750 to 4000 mg per day, most preferably 1000 to 3000 mg per day. DHA is preferably administered in an amount of 500 to 5000 mg per day, more preferably 750 to 4000 mg per day, most preferably 1000 to 3000 mg per day. If at all, EPA is preferably administered in an amount of 500 to 5000 mg per day, more preferably 750 to 4000 mg per day, most preferably 1000 to 3000 mg per day. These amounts of EPA apply if it is used alone or in combination with DHA.

In terms of unit dosage, the proportion of DHA+EPA+DPA (preferably DHA+EPA) of the total fatty acids is preferably 5 to 95 weight %, more preferably 10 to 80 weight %, most preferably 15 to 70 weight %. The present composition preferably comprises 5 to 95 weight % DHA based on total fatty acids, preferably 10 to 75 weight % DHA based on total fatty acids, more preferably 10 to 60 weight % DHA based on total fatty acids. The present composition preferably comprises 5 to 95 weight % EPA based on total fatty acids, preferably 10 to 75 weight % EPA, most preferably 15 to 60 weight %, based on total fatty acids.

The ratio of the weights of DHA to EPA is preferably larger than 1, more preferably 2:1 to 10:1, more preferably 3:1 to 8:1. The above-mentioned ratios and amounts take into account and optimize several aspects, including taste (too high LCP levels reduce taste, resulting in a reduced compliance), balance between DHA and precursors thereof to ensure optimal effectiveness while maintaining low-volume formulations.

Sources of DHA possible sources of DHA: tuna oil, (other) fish oils, DHA rich alkyl esters, algae oil, egg yolk, or phospholipids enriched with n-3 LCPUFA e.g. phosphatidylserine-DHA.

### ALA/LA

It is preferred that the alpha-linolenic acid [ALA] content of the composition is maintained at low levels. The inventors believe that due to the inflammatory nature of neurotrauma, excess supply of highly unsaturated fatty acids increases the risk of further damage to injury tissue due to the effect of peroxidized PUFAs, even though it has been observed that in vivo supply of α-linolenic acid is neuroprotective in neurotrauma (King et al. J. Neurosci. (26) 17:4672-4680). The ALA concentration is preferably maintained at levels less than 2.0 weight %, more preferably below 1.5 weight %, particularly below 1.0 weight %, calculated on the weight of all fatty acids.

Linoleic acid [LA] concentrations can be maintained at normal levels, i.e. between 20 to 30 weight %, although in one embodiment the LA concentration is also significantly reduced to an amount of <15 g/100 g fatty acids and even less than 10 weight %. The LA concentrations are preferably at least 1 weight % of the fatty acids.

In one embodiment, the weight ratio ω-3/ω-6 in the composition of the invention is preferably in the range 0.3 to 7, preferably in the range 1.4:1 to 5.9:1, more preferably in the range 3:1 to 5.5:1, most preferably 3:1 to 5:1, in particular less than 5:1. The amount of ω-6 LCPUFAs is preferably less than 50, preferably 5 to 40, more preferably 8 to 30 weight % of the fatty acids in the formula.

### MCT

In one embodiment, the composition contains less than 5 weight %, preferably less than 2 weight % of fatty acids of less than 14 carbon atoms.

Medium chain fatty acids [MCT] are defined to be linear or branched saturated carboxylic acids having six (C6:0), seven (C7:0), eight (C8:0), nine (C9:0) or ten (C10:0) carbon atoms. The amount of MCTs are preferably lower than 2 weight %, more preferably lower than 1.5 weight %, most preferably lower than 1.0 weight % of the total fatty acids. In one embodiment, the sum of the medium chain fatty acids C6:0+C7:0+C8:0 over the sum of C9:0 and C10:0 is less than 2:1, more preferably less than 1.8:1, most preferably less than 1.6:1.

### Choline

The present composition preferably contains choline, a choline salt and/or choline ester (e.g. C₁₋₆ alkanoate ester). The choline salt is preferably selected from choline chloride, choline bitartrate, or choline stearate. The choline ester is preferably selected from a phosphatidylcholine and lyso-phosphatidyl choline. The present method preferably comprises the administration of more than 50 mg choline per day, preferably 80 to 3000 mg choline per day, more preferably 100 to 2000 mg choline per day, most preferably 150 to 1000 mg choline per day. The present composition preferably comprises 80 mg to 3000 gram choline per 100 ml of the liquid composition, preferably 100 mg to 2000 mg choline per 100 ml, preferably 200 to 1000 mg choline per 100 ml composition, most preferably 200 mg to 600 mg choline per 100 ml. The above numbers are based on choline, the amounts of choline equivalents or sources can be calculated taking the molar equivalent to choline into account.

### B Vitamins

The present combination preferably comprises at least one B complex vitamin. The vitamin B is selected from the group of vitamin B1 (thiamine), vitamin B2 (riboflavin), vitamin B3 (niacin or niacinamide), vitamin B5 (pantothenic acid), vitamin B6 (pyridoxine, pyridoxal, or pyridoxamine, or pyridoxine hydrochloride), vitamin B7 (biotin), vitamin B9 (folic acid or folate), and vitamin B12 (various cobalamins). Functional equivalents are encompassed within these terms.

Preferably, at least one vitamin B is selected from the group of vitamin B6, vitamin B12 and vitamin B9. Preferably the present composition comprises at least two selected from the group consisting of vitamin B6, vitamin B12 and vitamin B9. In particular, good results have been achieved with a combination comprising vitamin B6, vitamin B12 and vitamin B9.

The vitamin B is to be administered in an effective dose, which dose depends on the type of vitamin B used. As a rule of thumb, a suitable minimum or a maximum dose may be chosen based on known dietary recommendations, for instance as recommended by Institute of Medicine (IOM) of the U.S. National Academy of Sciences or by Scientific Committee on Food (a scientific committee of the EU), the information disclosed herein and optionally a limited amount of routine testing. A minimum dose may be based on the estimated average requirement (EAR), although a lower dose may already be effective. A maximum dose usually does not exceed the tolerable upper intake levels (UL), as recommended by IOM.

If present in the nutritional composition or medicament, the vitamin B6 is usually present in an amount to provide a daily dosage in the range of 0.1 to 100 mg, in particular in the range of 0.5 to 25 mg, more in particular in the range of 0.5 to 5 mg. The present composition preferably comprises 0.1 to 100 mg vitamin B6 per 100 g (liquid) product, more preferably 0.5 to 5 mg vitamin B6 per 100 g (liquid) product, more preferably 0.5 to 5 mg vitamin B6 per 100 g (liquid) product.

If present in the nutritional composition or medicament, the vitamin B12 is usually present in an amount to provide a daily dosage in the range of 0.5 to 100 μg, in particular in the range of 1 to 10 μg, more in particular in the range of 1.5 to 5 μg. The present composition preferably comprises 0.5-100 μg vitamin B12 per 100 g (liquid) product, more preferably 1 to 10 μg vitamin B12 per 100 g (liquid) product, more preferably 1.5 to 5 μg vitamin B12 per 100 g (liquid) product. The term “vitamin B12” incorporates all cobalamin equivalents known in the art.

If present in the nutritional composition or medicament, the vitamin B9 is usually present in an amount to provide a daily dosage in the range of 50 to 5000 μg, in particular in the range of 100 to 1000 μg, more in particular in the range of 200 to 800 μg. The present composition preferably comprises 50 to 5000 μg folic acid per 100 g (liquid) product, more preferably 100 to 1000 μg folic acid per 100 g (liquid) product, more preferably 200 to 800 μg folic acid per 100 g (liquid) product. Folates include folic acid, folinic acid, methylated, methenylated and formylated forms of folates, their salts or esters (e.g. C₁₋₆ alkyl ester), as well as their derivatives with one or more glutamic acid, and all in either reduced or oxidized form.

### Phospholipids

It is preferred to incorporate at least one phospholipid in the composition. The term “phospholipid” excludes PC that is already accounted for in the choline fraction. The present composition preferably comprises at least one phospholipid in an amount of 0.01 to 1 gram per 100 ml, more preferably between 0.05 and 0.5 gram per 100 ml, most preferably 80 to 600 mg per 100 ml. The at least one phospholipid is preferably provided for using lecithin.

### Vitamins C, E

Vitamin C, or a functional equivalent thereof, may be present in an amount to provide a daily dosage in the range of 20 to 2000 mg, in particular in the range of 30 to 500 mg, more in particular in the range of 75 to 150 mg. In one embodiment, vitamin C, or a functional equivalent thereof, is present in an amount in the range of 20 to 2000 mg, in particular in the range of 30 to 500 mg, more in particular in the range of 75 to 150 mg per 100 ml of the composition.

Tocopherol and/or an equivalent thereof (i.e. a compound having vitamin E activity) may be present in an amount to provide a daily dosage in the range of 10 to 300 mg, in particular in the range of 30 to 200 mg, more in particular in the range of 35 to 100 mg, to prevent oxidative damage to the injury site resulting from dietary PUFA. In one embodiment, tocopherol and/or equivalent is present in an amount in the range of 10 to 300 mg, in particular in the range of 30 to 200 mg, more in particular in the range of 35 to 100 mg per 100 ml of the composition. The term “tocopherol and/or an equivalent thereof”, as used in this description, comprises tocopherols, tocotrienols, pharmaceutical and/or nutritional acceptable derivatives thereof and any combination thereof. The above numbers are based on tocopherol equivalents, recognized in the art.

### Selenium

The present composition preferably contains selenium. The antioxidant activity of selenium advantageously prevents and/or inhibits damages to the brain areas. Preferably the present method provides the administration of a composition comprising 0.01 and 5 mg selenium per 100 ml liquid product, preferably 0.02 and 0.1 mg selenium per 100 ml liquid product. The amount of selenium administered per day is preferably more than 0.01 mg, more preferably 0.01 to 0.5 mg.

In view of the above, the composition according to the invention preferably comprises uridine and/or UMP, the omega-3 PUFAs DHA and EPA, choline, phospholipids, folic acid, vitamin B12 and vitamin B6, in any of the aforementioned forms, equivalents or derivatives. The composition preferably comprises uridine and/or UMP, the omega-3 PUFAs DHA and EPA, choline, phospholipids, folic acid, vitamin B12, vitamin B6, vitamin C, vitamin E, and selenium, in any of the aforementioned forms, equivalents or derivatives.

In one embodiment, the composition according to the invention comprises per daily dosage or per 100 ml of liquid (preferably water):
100-500 mg, preferably 200-400 mg, more preferably about 300 mg EPA,
900-1500 mg, preferably 950-1300 mg, more preferably about 1200 mg DHA,
50-600 mg, preferably 60-200 mg, more preferably about 106 mg phospholipids,
200-600 mg, preferably 300-500 mg, more preferably about 400 mg choline,
400-800 mg, preferably 500-700 mg, more preferably about 625 mg UMP (uridine monophosphate),
20-60 mg, preferably 30-50 mg, more preferably about 40 mg vitamin E (alpha-TE),
60-100 mg, preferably 60-90 mg, more preferably about 80 mg vitamin C,
40-80 μg, preferably 45-65 μg, more preferably about 60 μg selenium,
1-5 μg, preferably 2-4 μg, more preferably about 3 μg vitamin B12,
0.5-3 mg, preferably 0.5-2 mg, more preferably about 1 mg vitamin B6, and
200-600 μg, preferably 300-500 μg, more preferably about 400 μg folic acid.

The compositions as described above can be used as a nutritional therapy, nutritional support, as a medical food, as a food for special medical purposes or as a nutritional supplement. Such product can be consumed at one, two or three servings of 125 mL per day during recovery and/or rehabilitation in the context of the impairments according to the invention.

Preferably, the composition is enterally administered to the subject according to the invention for at least one time per day for a period of at least 3 weeks, preferably at least 4 weeks, more preferably at least 5 weeks, particularly at least 6 weeks, most preferably at least 12 weeks.

### Examples

### Example 1a: Liquid Product Containing Per 125 ml Serving

| | |
|---|---|
| Fat, g 4.9 | Vitamin E (alpha-TE), mg 40 |
| EPA, mg 300 | Vitamin C, mg 80 |
| DHA, mg 1200 | Selenium, μg 60 |
| Phospholipids, mg 106 | Vitamin B12, μg 3 |
| Choline, mg 400 | Vitamin B6, mg 1 |
| UMP (uridine monophosphate), mg 625 | Folic acid, μg 400 |

Abbreviations:
EPA, eicosapentaenoic acid;
DHA, docosahexaenoic acid;
TE, tocopherol equivalents;

### Example 1b: Liquid Product Containing Per 125 ml Serving

| | |
|---|---|
| Energy, kcal 125 | Calcium, mg 100 |
| Protein, g 3.8 | Phosphorus, mg 87.5 |
| Carbohydrate, g 16.5 | Magnesium, mg 25.0 |
| Fat, g 4.9 | Iron, mg 2 |
| EPA, mg 300 | Zinc, mg 1.5 |
| DHA, mg 1200 | Iodine, μg 16.3 |
| Phospholipids, mg 106 | Manganese, mg 0.41 |
| Choline, mg 400 | Copper, μg 225 |
| UMP (uridine monophosphate), mg 625 | Molybdenum, μg 12.5 |
| Vitamin E (alpha-TE), mg 40 | Chromium, μg 8.4 |
| Vitamin C, mg 80 | Vitamin A, μg 200 |
| Selenium, μg 60 | Thiamin (B1), mg 0.19 |
| Vitamin B12, μg 3 | Riboflavin (B2), mg 0.20 |
| Vitamin B6, mg 1 | Niacin (B3), mg NE 2.25 |
| Folic acid, μg 400 | Pantothenic acid (B5), mg 0.66 |
| Sodium, mg 125 | Vitamin D, μg 0.88 |
| Potassium, mg 187.5 | Biotin, μg 5.0 |
| Chloride, mg 156.3 | Vitamin K, μg 6.6 |

Abbreviations:
EPA, eicosapentaenoic acid;
DHA, docosahexaenoic acid;
TE, tocopherol equivalents;
NE, niacin equivalents.

1.-14. (canceled)

15. A method for treating, stabilizing or slowing down brain glucose metabolism deficit in a subject in need thereof, the method comprising administering to the subject a composition comprising (i) one or more of uridine and cytidine, or salts, phosphates, acyl derivatives or esters thereof, and (ii) a lipid fraction comprising at least one of docosahexaenoic acid (22:6; DHA), eicosapentaenoic acid (20:5; EPA) and docosapentaenoic acid (22:5; DPA), or esters thereof.

16. The method according to claim 15, further comprises identifying a subject and/or monitoring brain glucose metabolism of the subject using Positron Emission Tomography with 18F-fluorodeoxyglucose (18F-FDG-PET).

17. The method according to claim 15, wherein the subject suffers from cerebral glucose metabolism deficit.

18. The method according to claim 17, wherein the subject suffers from subjective memory concerns or subjective memory complaints (SMI) and/or has a familial history of dementia or AD, the subject not suffering from MCI or prodromal dementia, prodromal AD.

19. The method according to claim 15, wherein the subject suffers from mild cognitive impairment (MCI) and/or a weight ratio of abeta-42/Phospho-tau-181 of less than 6.5 in CSF.

20. The method according to claim 19, wherein the subject suffering from MCI has a minimental state examination (MMSE) score of 20-26.

21. The method according to claim 15, wherein the subject is a prodromal AD or dementia subject.

22. The method according to claim 21, wherein the subject has dementia exhibiting a level of more than 350 ng Total-tau per litre cerebrospinal fluid (CSF).

23. The method according to claim 15, wherein the composition further comprises at least one of:
(iii) choline, or salts or esters thereof; or
(iv) at least one vitamin B selected from the group of vitamin B6, vitamin B12 and vitamin B9, or equivalents thereof.

24. The method according to claim 15, the composition further comprising at least one vitamin B selected from the group of vitamin B6, vitamin B12 and vitamin B9, or equivalents thereof.

25. The method according to claim 24, wherein the composition comprises vitamin B6, B9 and B12.

26. The method according to claim 15, wherein the composition comprises 9 to 300 mg/100 kJ DHA+EPA+DPA, per day.

27. The method according to claim 15, wherein the composition comprises 9 to 300 mg/100 kJ DHA+EPA, per day.

28. The method according to claim 15, wherein the composition comprises 1.5 to 130 mg/100 kJ of one or more of uridine, cytidine, or salts, phosphates or esters thereof, calculated as uridine and cytidine.

29. The method according to claim 15, wherein the composition further comprises 1 to 300 mg/100 kJ of choline, or salts or esters thereof, calculated as choline.

30. The method according to claim 15, wherein the composition further comprises one or more of vitamin C or its equivalents, vitamin E or its equivalents, and/or selenium.

31. The method according to claim 15, wherein the composition further comprises at least one phospholipid.

32. The method according to claim 15, the composition being aqueous and comprising, per daily dosage or per 100 ml of liquid:
100-500 mg EPA,
900-1500 mg DHA,
50-600 mg phospholipids,
200-600 mg choline,
400-800 mg UMP (uridine monophosphate),
20-60 mg vitamin E (alpha-TE),
60-100 mg vitamin C,
0-80 μg selenium,
1-5 μg vitamin B12,
0.5-3 mg vitamin B6, and
200-600 μg folic acid.
